(19) United States
(12) Patent Application Publication
(10) Pub. No.: US 2020/0101193 A1
(43) Pub. Date: Apr. 2, 2020

(54) IMPLANT AND METHOD OF PRODUCTION THEREOF

(71) Applicant: DOT GmbH, Rostock (DE)

(72) Inventors: Axel BAUMANN, Rostock (DE); Henry DEMPWOLF, Rostock (DE); Marcus KRAWUTSCHKE, Rostock (DE)

(21) Appl. No.: 16/567,764

(22) Filed: Sep. 11, 2019

(30) Foreign Application Priority Data
Sep. 27, 2018 (DE) 10 2018 123 874.6

(51) Int. Cl. A61L 27/06; C23C 14/06

(52) U.S. Cl. CPC A61L 27/06; A61L 2430/38; A61L 2430/24; C23C 14/0641

(57) ABSTRACT

An implant for insertion into a human or animal body including at least a substrate made of a metallic material and a surface formed on the substrate. The surface is completely or partially provided with a hard material coating. The hard material coating has a basic layer of TiN or TiNbN applied by means of a PVD method and an outer layer of TiAlN applied onto the basic layer by means of a PVD method. A method of production of the implant by using a PVD method.

The present invention relates to an implant for insertion into a human or animal body having at least a substrate made of a metallic material and a surface formed on the substrate that is completely or partially provided with a hard material coating. Furthermore, the invention relates to a method of producing the implant.

Implants of the above described type and methods of production thereof are known. Such implants are used, for example, in the form of hip and knee joint endoprostheses. Another field of application is spinal prosthetics, in particular intervertebral disc prosthetics.

Implants made of a zirconium-niobium alloy in which, in a heat treatment process, defined surfaces are converted into a wear-reducing zirconium oxide ceramic are commercially available. This ceramic layer is also referred to as “oxinium surface”. However, the implants produced this way are only usable to a limited extent and relatively expensive. In addition, zirconium alloys have only very poor tribological properties requiring an immediate replacement of the implant in the case of a layer failure.

EP1 916 007 shows a medical implant having a metal substrate and a coating formed on the substrate that comprises an intermediate layer and an outer layer made of aluminum oxide. The implant can be produced by a method in which an intermediate layer made of another material than aluminum oxide is applied onto the substrate surface and an outer layer made of aluminum oxide is deposited by physical vapor deposition (PVD) on at least a portion of the intermediate layer. As an intermediate layer, a material selected from the group consisting of titanium aluminum nitride (TiAlN), chromium aluminum nitride (CrAlN), aluminum nitride (AlN), titanium carbonitride (TiCN), titanium nitride (TiN), chromium oxide (Cr₂O₃), titanium aluminide (TiAl), chromium nitride (CrN) and combinations thereof can be used.

DE10 2006 039 329 B3 discloses an implant for insertion into a human or animal body in which the articular surface is partially or completely covered with a wear-reducing hard material coating. The hard material coating can comprise an outer top layer, for example made of zirconium nitride (ZrN), and an intermediate layer for reducing tensions between the hard material coating and the implant material. The intermediate layer can be formed as a multilayer system having at least two different layers lying alternately on top of each other. For example, one of the two layers can be a chromium nitride (CrN) layer and the other of the two different layers can be a chromium carbonitride (CrCN) layer, preferably resulting in a layer system comprising five layers whose outer layers are formed by chromium nitride (CrN) layers. Alternatively, titanium nitride (TiN) layers and titanium carbonitride (TiCN) layers can also be provided as different layers.

The coatings are intended to reduce or prevent the escape of metal ions such as cobalt, chromium, molybdenum and/or nickel ions from the implant material, thus reducing corrosion of the implant or wear due to flaking of the hard material coating. Moreover, tolerability of the implant can be improved, and the occurrence of allergic reactions caused by the escape of metal ions from the implant material can be prevented by the coating.

From EP 2 569 022 B1 a substrate for joints of orthopedic implants is known, with at least one of the sliding surfaces formed of non-ferrous material, in particular of cobalt, chromium, molybdenum alloys, and having a coating made of nitride layers. The coating contains niobium nitride nanolayers and chromium nitride nanolayers and is protected by a chromium nitride microlayer. The niobium nitride nanolayers can alternate with the chromium nitride nanolayers.

WO 2012/003899 A1 relates to a medical product having an antibacterial hard material coating with biocide applied onto a basic body. This hard material coating comprises at least an inner layer and an outer layer, with the biocide concentration in the outer layer being substantially constant and greater than the biocide concentration in the inner layer, and the biocide concentration in the inner layer being greater than or equal to 0.2 at %. In the embodiment silver-doped TiN layers are disclosed.

However, the known coatings of implants require multilayer structures, and their production is thus relatively expensive. In addition, the provision of a plurality of different layers can lead to local element formation and undesired mechanical tensions between the coating and the implant material and/or to internal tensions in the hard material coating, thus affecting the adhesive strength of the coating. In addition, coatings having an outer layer made of aluminum oxide are relatively rough and tend towards premature wear of a joint partner.

The deposition of TiAlN layers on a substrate, in particular on a hard material substrate, using methods of physical vapor deposition (PVD methods) is extensively described in the state of the art. Regarding this, reference can be made, for example, to DE 10 2012 107 129 A1, EP 1 273 679 A1 and DE 196 14 557 A1.

It is the object of the present invention to provide an implant of the above described type having an inexpensive wear protection layer. In particular, the coating is intended to have a good adhesive strength on the implant material and to ensure a sufficient lifespan of the implant.

This object is solved by an implant according to claim 1. Further advantageous embodiments are stated in the sub-claims, which can optionally be combined with each other.

The implant according to the present invention for insertion into a human or animal body comprises at least a substrate made of a metallic material and a surface formed on the substrate. The surface is completely or partially provided with a hard material coating. The implant is characterized in that the hard material coating comprises a basic layer of TiN or TiNbN applied by means of a PVD method and an outer layer of TiAlN applied onto the basic layer by means of a PVD method.

In particular, the substrate surface can be formed at least partially in the form of an artificial articular surface, with the articular surface being completely or partially, preferably completely, provided with the hard material coating.

Furthermore, the invention relates to a method of producing the implant according to claim 11 and the use of the implant produced according to said method as a hip joint prosthesis, knee joint prosthesis, ankle joint prosthesis or shoulder joint prosthesis, vertebral body replacement implant or intervertebral disc prosthesis.

By using the PVD method, a wear-resistant hard material coating in the form of a two-layer system having a basic layer of TiN or TiNbN and an out layer of TiAlN directly adjacent to the basic layer can be produced in a quick and inexpensive manner. At the same time, the mechanical tensions between the hard material coating and the substrate and/or internal tensions in the hard material coating can be reduced by the layer system according to the present invention.

According to the present invention, the TiAlN layer is present as an outer layer of the hard material coating. Thus, no further layer is envisaged on the TiAlN layer. In particular, the TiAlN layer is in direct contact with body tissue and/or a contact surface on a joint partner.

The two-layer system made of TiNbN and TiAlN or TiN and TiAlN is an excellent barrier for the passage of metal ions from the substrate into the body tissue. In addition, the TiAlN outer layer can be produced as a smooth and low-defect surface having an extremely low surface roughness, thus reducing wear by abrasion of the TiAlN layer or the contact surface of the joint partner adjacent to the TiAlN layer. Therefore, less foreign particles enter he body of the implant wearer and the danger of allergies can be significantly reduced. At the same time, the hard material coating acts as a corrosion protection for the coated articular surface.

Additionally, as the TiAlN outer layer has an excellent wettability for body fluids, the tribological properties of the articular surface are favorably influenced.

The layer system according to the present invention also shows a very good adhesive strength both on the substrate and within the layer system. Thus, the danger of failure of the coating by flaking under stress is significantly reduced.

Due to the high hardness of the TiAlN outer layer the coating can be produced in a low layer thickness of only a few micrometers. In so doing, production costs can be saved without affecting the performance of the implant. Moreover, a high layer hardness also indicates a resistance of the articular surface to three-body wear and an improved scratch resistance.

According to a preferred embodiment the TiAlN outer layer has a composition of Ti₁₋ₓAlₓN, with 0.2≤x≤0.8, preferably 0.5≤x≤0.7, each with the exception of inevitable impurities. A TiAlN layer having the described composition can be easily produced by means of PVD methods. The TiAlN layer has a good adhesive strength on the TiNbN basic layer and a high hardness.

Preferably, the TiAlN layer is a monolithic layer. Monolithic means that within the TiAlN layer no alternating layers having a different layer composition are present at the respective interfaces. However, the TiAlN layer can have an aluminum gradient with an aluminum proportion increasing outward from the substrate surface towards the outer surface of the implant to improve adhesion on the TiN or TiNbN basic layer. Furthermore, the hardness and wear resistance of the TiAlN layer can be further improved by a higher aluminum proportion at the outer surface of the implant.

Preferably, the basic layer is a TiNbN layer, in particular a monolithic layer, having a composition of Ti₁₋ₓNbₓN and 0.1≤x≤0.4, with the exception of inevitable impurities.

The Nb in the TiNbN layer can be partially replaced by Ta, preferably in a proportion of up to 30 at %, preferably of up to 10 at %, and particularly preferably of up to 5 at %, without affecting the layer properties. It is known that Nb and Ta are socialized with each other, and a complete separation of the elements can only be achieved with high costs due to the very similar properties of Nb and Ta. However, Ti and Nb are preferably present in the TiNb target employed as pure elements, with the exception of inevitable impurities.

Preferably, the TiNbN layer is directly applied onto the articular surface of the metallic substrate. Directly means that no further functional layers are present between the articular surface and the TiNbN layer, with the exception of an adhesion layer made of titanium or a titanium alloy with a thickness of up to 500 nm that promotes adhesion of the TiNbN layer on the metallic substrate. The provision of such adhesion layers is generally known in PVD methods.

According to a preferred embodiment the hard material coating thus consists of the basic layer, the TiAlN outer layer and optionally the adhesion layer made of titanium or a titanium alloy.

The substrate can be made of any metallic material suitable for the production of implants that are incorporated into the human body or an animal body. Known suitable materials are, for example, steel, titanium and titanium alloys as well as cobalt and cobalt alloys.

Preferably, the material contains cobalt or a cobalt alloy. It is advantageous when the cobalt alloy is a cobalt-chromium-molybdenum alloy. Preferably, it is a CoCr29Mo6 alloy.

Suitable titanium alloys include but are not limited to a titanium-aluminum-vanadium alloy such as Ti-3A-I2.5V or Ti-6Al-4V.

A steel suitable as a substrate material for implants is, for example, stainless steel 1.4301 (X5CrNi18-10), 1.4404 (X2CrNiMo18-14-3) and 1.4435 (X2CrNiMo18-14-3).

The total thickness of the hard material coating is preferably from 2 to 10 μm, more preferably from 3 to 8 μm and particularly preferably from 4 to 7 μm. The layer thickness of the TiN or TiNbN basic layer is preferably in a range of 1 to 5 μm, more preferably of 1 to 4 μm. Preferably, the TiAlN outer layer has a layer thickness of 1 to 6 μm, particularly preferably of 2 to 5 μm.

Despite the comparably low layer thickness the hard material coating has a good wear resistance. Coatings having a higher layer thickness are less stable and tend to flake off the substrate.

By applying the hard material coating using the PVD method layers having a low surface roughness and favorable tribological properties can be provided. Preferably, the mean surface roughness Ra is below 0.05 μm, more preferably below 0.03 μm and particularly preferably in a range of 0.01 μm to 0.03 μm. The mean surface roughness Ra is measured according to DIN EN ISO 4287.

Known polishing methods such as grinding or brushing with correspondingly hard and fine materials are suitable to further smooth the surface of the implant following deposition of the layers. However, by suitable selection of the deposition parameters in the PVD method a low mean surface roughness Ra can be obtained even directly after deposition of the TiAlN layer. Thus, a subsequent smoothing of the surface can be omitted.

Compared to conventional implant coatings having a TiNbN outer layer the wear resistance of the hard material coating is substantially improved. Particularly preferably, the hard material coating having the TiAlN outer layer has a microhardness HV according to Vickers of 3200 to 3600 HV (test force 0.080 N from Martens hardness) that is about 40% higher than the hardness of a TiNbN layer.

The adhesive strength of the hard material coating can be determined in a Rockwell test. In the Rockwell test a diamond cone is impressed with a defined force into the layer surface. In the surrounding of the hardness impression the layer is damaged, which can be seen under the microscope as crack networks or layer openings in the edge region of the impression. Flaking around the impression can be evaluated by using DIN EN ISO 26443 with digital image evaluation of the flaked off surface areas. According to DIN/ISO 26443 the adhesive strength of the hard material coating in the Rockwell test results in class 1 at most.

According to a preferred embodiment of the invention it can be envisaged that the implant is an artificial hip or knee joint, an artificial ankle joint, an artificial shoulder joint, a vertebral body replacement implant or an artificial intervertebral disc prosthesis.

Preferably, the substrate surface is formed at least partially in the form of an artificial articular surface, with the articular surface being completely or partially, preferably completely, provided with the hard material coating. For example, the articular surface can form a joint ball or a joint socket of a hip joint prothesis, an artificial condyle, a tibia plate of a knee joint prosthesis or a contact element of an intervertebral disc prosthesis.

A method of producing the implant according to the present invention for insertion into a human or animal body comprises the provision of a substrate made of a metallic material, with the substrate having a surface that can be formed at least partially in the form of an artificial articular surface, and the application of a hard material coating onto the entire surface or part of the surface by means of a PVD method. The method according to the present invention is characterized in that the hard material coating is applied by applying a basic layer made of TiN or TiNbN onto the surface and subsequently applying a TiAlN layer onto the basic layer, with the TiAlN layer forming the outer layer of the hard material coating.

By applying the hard material coating by means of a PVD method particularly good adhesion of the hard material coating on the substrate can be achieved. The TiN or TiNbN basic layer and the TiAlN outer layer are preferably applied without interruption in a single coating cycle.

Preferably, a TiNbN basic layer is applied onto the articular surface of the substrate by using a Ti—Nb target with 70 at % titanium and 30 at % niobium.

For example, the TiAlN outer layer can be applied by using a Ti—Al target with 33 at % titanium and 67 at % aluminum.

The PVD method can be performed as magnetron sputtering, cathodic arc deposition (Arc-PVD), ion plating, electron beam evaporation or laser ablation. Preferred PVD methods comprise pulsed and non-pulsed magnetron sputtering, HF magnetron sputtering and alternating current magnetron sputtering as well as pulsed and non-pulsed cathodic arc deposition. Nitrogen is supplied into the reaction space of the PVD method to generate nitrides. PVD devices for the application of hard material coatings are commercially available.

The subsequent description of preferred embodiments of the invention serves as a detailed explanation and is not to be understood in a limiting sense.

Implants according to the present invention for insertion into a human or animal body can be formed, for example, in the form of knee joint prostheses, hip joint prostheses, ankle joint prostheses, shoulder joint prostheses or intervertebral disc prostheses. However, this list is not final.

The implant comprises at least a substrate made of a metallic material and a surface formed on the substrate that can be formed in the form of an artificial articular surface. The surface, in particular the articular surface, is completely or partially, preferably completely, provided with a hard material coating.

According to the present invention, the hard material coating comprises a basic layer of TiN or TiNbN applied onto the substrate surface by means of a PVD method, and an outer layer of TiAlN applied onto the basic layer by means of a PVD method. Preferably, the hard material coating consists of the TiN or TiNbN basic layer and the TiAlN outer layer. Optionally, an adhesion layer made of titanium or a titanium alloy can be provided between the substrate and the TiN or TiNbN basic layer.

### PRODUCTION OF A HARD MATERIAL COATING ACCORDING TO THE PRESENT INVENTION

In the embodiment described here a substrate made of a cobalt alloy CoCr29Mo6 is provided with the hard material coating according to the present invention.

Initially, a TiNb adhesion layer having a thickness of 0.5 μm was deposited onto the substrate surface by means of cathodic arc deposition. A hard material coating according to the present invention with a total layer thickness of 5.5 μm was deposited onto this adhesion layer, again by means of cathodic arc deposition.

The TiNbN basic layer was deposited from a Ti—Nb mixed target having a Ti:Nb ratio of 70:30 (at %) at a substrate bias of 100 to 200 V and a nitrogen pressure of 2 to 4 Pa. The deposition temperature was about 400 to 500° C. A TiNbN layer with a layer thickness of 1 μm was obtained.

A TiAlN layer was deposited from a Ti—Al mixed target with a Ti:Al ratio of 45:55 at % directly onto the TiNbN layer by means of cathodic arc deposition. The substrate bias was between 50 and 130 V at a nitrogen pressure of 1 to 4 Pa nitrogen. The deposition temperature was about 400 to 500° C. A TiAlN layer with a layer thickness of 5 μm was obtained.

### Production of a Comparative Coating

A monolithic TiNbN layer with a layer thickness of 5.5 μm was produced on the surface of a substrate made of CoCr29Mo6 by means of cathodic arc deposition. A Ti—Nb mixed target having a Ti:Nb ratio of 70:30 (at %) at a substrate bias of 100 to 200 V and a nitrogen pressure of 2 to 4 Pa was used. The deposition temperature was about 400 to 500° C.

Various measurements were performed on the coatings thus obtained to determine the surface roughness, the layer thickness, the hardness and the adhesive strength. The results are shown in the table below.

TABLE
Layer properties

| | | | |
|---|---|---|---|
| Parameter | Process | TiNbN—TiAlN | TiNbN |

| Mean | Tactile measurement | 0.02 | μm | ≤0.05 | μm |
|---|---|---|---|---|---|
| roughness | according to | | | | |
| Ra | DIN EN ISO 4287 | | | | |
| Layer | Calotte grinding | 5.3 | μm | 4.5 | μm |
| thickness | according to | | | | |
| | DIN EN 1071-2 | | | | |
| Hardness | Recording | 3384 | HV | 2400 | HV |
| | microhardness | | | | |
| | measurement | | | | |
| | according to Vickers | | | | |
| | (DIN EN ISO 14577- | | | | |
| | 4; test force 0.080 N) | | | | |
| Adhesive | Rockwell test | Class 1 at | | Class 1 at | |
| strength | (DIN EN ISO 26443) | most | | most | |
| Adhesive | Scratch test Lc2 | 49.23 | N | 49.30 | N |
| failure load | (DIN EN 1071-3) | | | | |

By means of the two-layer system according to the present invention a hard material layer for implants with good wear resistance properties that also meets the high requirements of joint prostheses with regard to tribological and mechanical properties can be produced inexpensively. The individual layers have a barrier function and, at the same time, show a high hardness and a high adhesive strength. Thus, the lifespan of the coated implant can be improved.

1. An implant for insertion into a human or animal body comprising at least a substrate made of a metallic material and a surface formed on the substrate, with the surface being completely or partially provided with a hard material coating, wherein the hard material coating comprises a basic layer of TiN or TiNbN applied onto the surface by means of a PVD method and an outer layer of TiAlN applied onto the basic layer by means of a PVD method.

2. The implant according to claim 1, wherein the surface of the substrate is formed at least partially in the form of an artificial articular surface, with the articular surface being completely or partially provided with the hard material coating.

3. The implant according to claim 1, wherein the TiAlN outer layer has a composition of Ti₁₋ₓAlₓN, wherein 0.2≤x≤0.8, with the exception of inevitable impurities.

4. The implant according to claim 3, wherein 0.50≤x≤0.7, with the exception of inevitable impurities.

5. The implant according to claim 2, wherein the TiAlN outer layer has a composition of Ti₁₋ₓAlₓN, wherein 0.2≤x≤0.8, with the exception of inevitable impurities.

6. The implant according to claim 1, wherein the TiAlN layer has an aluminum gradient with an aluminum proportion increasing outward from the substrate surface towards the outer layer of the implant.

7. The implant according to claim 1, wherein the basic layer is a TiNbN layer.

8. The implant according to claim 7, wherein the TiNbN layer has a composition of Ti₁₋ₓNbₓN and 0.1≤x≤0.4, with the exception of inevitable impurities.

9. The implant according to claim 1, wherein the hard material coating has an adhesion layer of titanium or a titanium alloy that is provided between the basic layer and the substrate surface, with the adhesion layer having a layer thickness of 0 to 500 nm.

10. The implant according to claim 1, wherein the hard material coating has a total thickness in a range of 2 to 10 μm.

11. The implant according to claim 10, wherein the total thickness of the hard material coating is in a range of 3 to 8 μm.

12. The implant according to claim 10, wherein the total thickness of the hard material coating is in a range of 4 to 7 μm.

13. The implant according to claim 1, wherein the basic layer has a layer thickness in a range of 1 to 5 μm.

14. The implant according to claim 13, wherein the layer thickness of the basic layer is in a range of 1 to 4 μm.

15. The implant according to claim 1, wherein the TiAlN outer layer has layer thickness of 1 to 6 μm.

16. The implant according to claim 15, wherein the layer thickness of the TiAlN outer layer is 2 to 5 μm.

17. The implant according to claim 1, wherein the hard material coating has one or more of the following properties:
a mean surface roughness Ra below 0.05 μm;
a microhardness HV according to Vickers of 3200 to 3600 HV at a test force of 0.080 N; and/or
a Rockwell adhesive strength according to DIN EN ISO 26443 of class 1 at most.

18. The implant according to claim 17, wherein the mean surface roughness Ra is in a range of 0.01 μm to 0.03 μm.

19. A method of producing an implant according to claim 1, comprising the following steps:
providing a substrate made of a metallic material, with the substrate having a surface; and
applying a hard material coating onto the entire surface or part of the surface by means of a PVD method,
characterized in that the hard material coating is applied by applying a basic layer made of TiN or TiNbN onto the substrate surface and subsequently applying a TiAlN layer onto the basic layer, with the TiAlN layer forming the outer layer of the hard material coating.

20. The method of claim 19, wherein the implant is a knee joint prosthesis, hip joint prosthesis, ankle joint prosthesis, shoulder joint prosthesis, vertebral body replacement implant or intervertebral disc prosthesis.
